(19) United States
(12) Patent Application Publication
(10) Pub. No.: US 2011/0124614 A1
(43) Pub. Date: May 26, 2011

(54) Methods And Compositions For The Treatment of Autoimmune Disorders

(76) Inventors: HALINA OFFNER, PORTLAND, OR (US); CHUNHE WANG, Carmel, IN (US); BABAK DEHGHANI, ROCKVILLE, MD (US)

(21) Appl. No.: 12/779,763

(22) Filed: May 13, 2010

Related U.S. Application Data
(63) Continuation of application No. 12/589,445, filed on Oct. 23, 2009, now abandoned.
(60) Provisional application No. 61/197,283, filed on Oct. 25, 2008.

(51) Int. Cl. A61K 31/47; A61K 31/565; A61P 29/00; A61P 37/06; A61P 25/00

(52) U.S. Cl. 514/171; 514/311

(57) ABSTRACT

Methods and compositions are provided which confer protection against autoimmune diseases without triggering intracellular estrogen receptors. Such methods and compositions limit the side effects of steroids while providing the benefits conferred by such medications through the activation of membrane estrogen receptors.

### CROSS REFERENCES TO RELATED APPLICATIONS

This application claims priority to U.S. Provisional Patent Application No. 61/197,283, filed Oct. 25, 2008, which is incorporated herein by reference in its entirety.

### STATEMENT REGARDING GOVERNMENT SPONSORED RESEARCH

Aspects of this work were supported by grants from the National Institutes of Health (NS45445 and NS49210); and National Multiple Sclerosis Society Grant RG3405. The United States government has certain rights in the subject matter.

### TECHNICAL FIELD

The present invention relates to compositions and methods for treatment of immune disorders, including autoimmune disorders.

### BACKGROUND

The anti-inflammatory effects of sex steroids and glucocorticoids have long been utilized to control allergy, asthma and autoimmune diseases. However, the mechanism(s) by which sex steroid hormones modulate immune cells or autoimmune processes is not completely understood. Estrogen and testosterone are believed to modulate the function of cells involved in the immune response. For example, sex steroids are believed to affect lymphoid or myeloid cell differentiation, cytokine production, Th polarization, nitric oxide production, MHC class II expression, and APC recruitment.

Symptoms of lymphocyte mediated autoimmune diseases in humans, e.g., multiple sclerosis (MS), rheumatoid arthritis (RA), Graves' disease, systemic lupus erythematosus (SLE) and Hashimoto's thyroiditis, and murine models of these diseases such as experimental autoimmune encephalomyelitis (EAE) and collagen-induced arthritis (CIA), alter during pregnancy leading to the theory that lower physiological amounts of 17β-estradiol (E2) are stimulatory to the immune system, while pharmacological doses or pregnancy levels of E2 alter and perhaps inhibit cell mediated immunity. Estrogens apparently play a role in this bias towards type 1 or type 2 cytokine profiles as demonstrated by model systems in which E2 levels are manipulated in vivo or in vitro. Pregnancy is characterized by a type 2 cytokine environment, with increased IL-4 and IL-10 and decreased pro-inflammatory cytokines. Thus, in Th1-mediated autoimmune diseases such as RA, thyroiditis, uveitis, psoriasis and MS, symptoms are often decreased during pregnancy when estrogen levels are high, yet flare during the postpartum period when estrogen levels decrease. (Nalbandian and Kovats, Curr. Med. Chem.—Immun., Endoc. & Metab. Agents, 2005, 5, 85-91)

Clinical application of estrogens in the treatment of autoimmune disease is limited by undesirable side effects, ranging from the triggering of breast and uterine cancer to the loss of appetite, rapid weight gain and fluid retention. Most “estrogenic” side effects are believed to be mediated through classic intracellular estrogen receptors (iERs) including ERα (Esr1) and ERβ (Esr2) (DSechering, K., et al. Curr. Med. Chem. 7:561-576 (2000)).

There is therefore a need in the art for achieving estrogen-like effects that reduce or eliminate symptoms of autoimmune diseases without activating classic iERs.

### SUMMARY OF EXEMPLARY EMBODIMENTS OF THE INVENTION

The present invention fulfills these needs and satisfies additional objects and advantages by providing methods and compositions for activating membrane estrogen receptors. Specifically, the present invention provides compositions and methods for generating steroidal effects without activating intracellular estrogen receptors (iERs) such as ERα and ERβ.

This invention is founded on the discovery that signaling through membrane estrogen receptors is sufficient to mediate protection against autoimmune disease without causing estrogenic side effects. Specifically, it is established that signaling through GPR30 is sufficient to mediate protection against autoimmune diseases, as exemplified by studies herein using generally accepted animal models of human autoimmune diseases, such as Experimental Autoimmune Encephalomyelitis (EAE) as a predictive model of drug, activity in human multiple sclerosis (MS) conditions.

Membrane estrogen receptors may be triggered by any means desired. In some embodiments, membrane estrogen agonists are used. Useful agonists are those that act without triggering intracellular estrogen receptors (iERs). Such agonists include, but are not limited to G-1, a cell-permeable, nonsteroidal, dihydroquinoline compound that acts as a high-affinity agonist for GPR30 and STX.

The methods and compositions of the present invention may additionally be used in the treatment of mammalian subjects including, but not limited to, humans and other mammalian subjects suffering from lymphocyte mediated autoimmune diseases including, but not limited to, multiple sclerosis (MS), rheumatoid arthritis (RA), Graves' disease, systemic lupus erythematosus (SLE) and Hashimoto's thyroiditis, and murine models of these diseases such as experimental autoimmune encephalomyelitis (EAE), collagen-induced arthritis (CIA), BCl transgene, APCS^{-/-}, or (NZB×NZW)F1, and MRL/lpr. In exemplary embodiments, experimental autoimmune encephalomyelitis is used as a model for testing the effects of signaling through GPR30.

These and other subjects are effectively treated, prophylactically and/or therapeutically, by administering to the subject an autoimmune treating effective amount of a membrane estrogen receptor agonist.

Within additional aspects of the invention, combinatorial formulations and methods are provided which employ an effective amount of a membrane estrogen receptor agonist compound in combination with one or more secondary or adjunctive active agent(s) that is/are combinatorially formulated or coordinately administered with the membrane estrogen receptor agonist compound to yield an auto immune treating effective response in the subject. Exemplary combinatorial formulations and coordinate treatment methods in this context employ the membrane estrogen receptor agonist in combination with one or more additional, secondary or adjunctive therapeutic agents. The secondary or adjunctive therapeutic agents used in combination with, e.g., G-1 in these embodiments may possess direct or indirect autoimmune treating activity alone or in combination with, e.g. G-1 or may exhibit other useful adjunctive therapeutic activity in combination with, e.g., G-1.

Useful adjunctive therapeutic agents in these combinatorial formulations and coordinate treatment methods include, for example, the secondary or adjunctive methods and compositions useful in the treatment of autoimmune diseases include, but are not limited to, immunoglobulins (e.g., a CTLA4Ig, such as BMS-188667; see, e.g., Srinivas et al., J. Pharm. Sci. 85(1):1-4, (1996), incorporated herein by reference); copolymer 1, copolymer 1-related peptides, and T-cells treated with copolymer 1 or copolymer 1-related peptides (see, e.g., U.S. Pat. No. 6,844,314, incorporated herein by reference); blocking monoclonal antibodies, transforming growth factor-β, anti-TNF α antibodies; and steroidal agents.

The invention achieves these objects and satisfies additional objects and advantages by activating membrane estrogen receptors without triggering intracellular estrogen receptors.

### BRIEF DESCRIPTION OF THE DRAWINGS

FIG. 1, panels A-C illustrate that GPR30 is required for E2-induced full protection against EAE.

FIG. 2, panels A-E illustrates that activation of GPR30 conferred substantial protection against clinical EAE in WTB6 mice.

FIG. 3 is a series of graphs indicating that G-1 treatment did not change the serum levels of E2, progesterone or testosterone and only slightly lowered corticosteroid.

FIG. 4 is a series of histological photographs indicating that G-1 treatment reduced CNS infiltration ((H&E) hematoxylin and eosin stain), demylenation ((LFB-PAS) Luxol fast blue/periodic acid-Schiff), axonal Loss ((NFLs) nerve fiber layer) and ongoing axonal damage (dephosphorylated NFLs).

FIG. 5 is a series of charts demonstrating that GPR30 enhanced Treg cell function by upregulating PD-1 (programmed death 1) expression levels.

FIG. 6 is a series of charts indicating that PD-1 is required for G-1 induced EAE protection and cytokine deviation.

### DETAILED DESCRIPTION OF EXEMPLARY EMBODIMENTS OF THE INVENTION

A broad range of mammalian subjects, including human subjects, are amenable to treatment using the formulations and methods of the invention. These subjects include, but are not limited to, human and other mammalian subjects presenting with lymphocyte mediated auto immune diseases including, but not limited to, multiple sclerosis (MS), rheumatoid arthritis (RA), Graves' disease, systemic lupus erythematosus (SLE) and Hashimoto's thyroiditis, and murine models of these diseases such as experimental autoimmune encephalomyelitis (EAE) and collagen-induced arthritis (CIA).

Within the methods and compositions of the invention, one or more agonists of membrane estrogen receptors disclosed herein are effectively formulated or administered as a stimulant of estrogen membrane receptors effective for treating autoimmune diseases. In exemplary embodiments, G-1 is used as an estrogen membrane receptor agonist for treating autoimmune disorders effective alone or in combination with one or more adjunctive therapeutic agent(s). In other exemplary embodiments, experimental autoimmune encephalomyelitis is used as a model for testing the effects of signaling through the G protein-coupled membrane estrogen receptor 30 (GPR30).

Estrogen is a steroid hormone involved in the regulation of a wide array of processes that include reproduction, sexual development, behavior, stress responses, bone integrity, neuroprotection, and cardiovascular health. Estrogen receptors (ER) such as ERα (Esr1) and ERβ (Esr2) are generally defined as ligand-dependent transcription factors located in the cytosol or nucleus.

Recently, estrogen and estrogen compounds have been shown to induce very rapid changes in physiological activity in certain cell types. These changes can occur within minutes and therefore cannot be mediated through the classical genomic mechanism that causes changes in gene transcription. Rapid responses to estrogen are thought to be mediated via a non-genomic mechanism that can include stimulation of nitric oxide production in pulmonary endothelial cells (Russell et al. Proc. Natl. Acad. Sci. U.S.A., 97, 5930, 2000), and increased activation of mitogen-activated protein kinase in neuronal cells (Singer et al., Journal of Neuroscience, 19, 2455, 1999), osteoblasts (Kousteni, et al., Cell, 104, 719, 2001), and breast cancer cells (Razandi et al., Molecular Endocrinology, 14, 1434, 2000). Such signaling can account for several 17β estradiol (E2) effects such as, but not limited to, inhibition of oxidative stress-induced apoptosis and upregulation of NGF in macrophages.

The rapid response to estrogen is believed to be mediated by membrane estrogen receptors (mERs). Membrane estrogen receptors participate in a wide range of complex functions influencing cell proliferation, death, and differentiation. These receptors can be found in a variety of locations including, but not limited to, membrane shells, rafts, and caveolae.

Membrane estrogen receptors are attached to membranes in a variety of ways. Some membrane estrogen receptors are pulled to the plasma membrane caveolins. Other evidence has shown that acylation of these receptors causes them to be imbedded into the membrane. Still other receptors traverse the membrane seven times like typical G proteins. (Watson and Gametchu Experimental Biology and Medicine 228:1272-1281 (2003)). Additional membrane receptors form complexes with G proteins, striatin, receptor tyrosine kinases (e.g. EGFR and IGF-1), and non-receptor tyrosine kinases. The present invention applies to all membrane estrogen receptors regardless of the means of embedding in the cell membrane.

Direct activation of membrane estrogen receptors confers the protection of administration of steroids without steroid side effects. This is done by avoiding the activation of intracellular estrogen receptors such as ERα (Esr1) and ERβ (Esr2) which are normally activated by the administration of steroids.

Activation of membrane estrogen receptors alters cytokine profiles. For example, secretion of IL-17, IL-4 and IL-2 may be decreased whereas secretion of IL-10, IL-6 and IFN-γ may be increased. Additionally, activation of membrane estrogen receptors may affect regulatory T cells. For example, activation of membrane estrogen receptors may enhance the suppressive activity of CD4⁺Foxp3⁺ Treg cells. Activation of membrane estrogen receptors may also upregulate programmed death 1 in Treg cells and programmed death 1 ligand (PD-L1) in antigen presenting cells.

Membrane estrogen receptors may be activated by any means possible, including through the use of targeted agonists such as G-1 or SIX.

The administration of a membrane estrogen receptor agonist protects against the disease and symptoms of the disease being targeted. For example, in experimental autoimmune encephalomyelitis (EAE), an animal model for multiple sclerosis, administration of the membrane receptor agonist G-1 ameliorated immune cell infiltration and demyelination in the central nervous system and reduced the level of axonal damage in spinal cord sections.

Membrane estrogen receptor agonists of the invention typically comprise an membrane estrogen receptor activating effective amount or unit dosage, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. An effective amount of a membrane estrogen receptor agonist will be readily determined by those of ordinary skill in the art, depending on clinical and patient-specific factors. Suitable effective unit dosage amounts of the active compounds for administration to mammalian subjects, including humans, may range from 10 to 1500 mg, 20 to 1000 mg, 25 to 750 mg, 50 to 500 mg, or 150 to 500 mg. In certain embodiments, the effective amount of the membrane estrogen receptor agonist may be selected within narrower ranges of, for example, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 10 to 25 mg, 30-50 mg, 75 to 100 mg, 100 to 250 mg, or 250 to 500 mg, are administered one, two, three, four, or five times per day. In more detailed embodiments, dosages of 50-75 mg, 100-200 mg, 250-400 mg, or 400-600 mg are administered once or twice daily. In alternate embodiments, dosages are calculated based on body weight, and may be administered, for example, in amounts from about 0.5 mg/kg to about 100 mg/kg per day, 1 mg/kg to about 75 mg/kg per day, 1 mg/kg to about 50 mg/kg per day, 2 mg/kg to about 50 mg/kg per day, 2 mg/kg to about 30 mg/kg per day or 3 mg/kg to about 30 mg/kg per day

The amount, timing and mode of delivery of compositions of the invention comprising a membrane estrogen receptor agonist will be routinely adjusted on an individual basis, depending on such factors as weight, age, gender, and condition of the individual, the acuteness of the autoimmune disorder and/or related symptoms, whether the administration is prophylactic or therapeutic, and on the basis of other factors known to effect drug delivery, absorption, pharmacokinetics, including half-life, and efficacy.

An effective dose or multi-dose treatment regimen for the instant membrane estrogen receptor agonist formulations will ordinarily be selected to approximate a minimal dosing regimen that is necessary and sufficient to substantially prevent or alleviate cellular proliferative disorders and diseases in the subject, and/or to substantially prevent or alleviate one or more symptoms associated with cellular proliferative disorders in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

Various assays and model systems can be readily employed to determine the therapeutic effectiveness of a compound. For example, animal models of autoimmune diseases such as experimental autoimmune encephalomyelitis, collagen induced arthritis, BC1-2 transgene, APCS^{-/-} or (NZB×NZW)F1, MRL/lpr may be used to determine the therapeutic effectiveness of a membrane estrogen receptor agonist. Effectiveness of the compositions and methods of the invention may be demonstrated by a decrease in the symptoms of autoimmune disorders. Such a decrease may be a decrease of 5%, 10%, 25%, 30%, 50%, 75%, 90% or more.

In additional aspects of the invention, combinatorial cellular proliferative disease treating formulations and coordinate administration methods are provided which employ an effective amount of membrane estrogen receptor agonist and one or more secondary or adjunctive agent(s) that is/are combinatorially formulated or coordinately administered with a membrane receptor agonist to yield a combined, multi-active agent autoimmune ameliorating composition or coordinate treatment method. Exemplary combinatorial formulations and coordinate treatment methods in this context employ the membrane estrogen receptor agonist in combination with the one or more secondary agents that is/are useful for treatment or prophylaxis of the targeted (or associated) disease, condition and/or symptom(s) in the selected combinatorial formulation or coordinate treatment regimen. For most combinatorial formulations and coordinate treatment methods of the invention, a membrane estrogen receptor agonist is formulated, or coordinately administered, in combination with one or more secondary or adjunctive therapeutic agent(s), to yield a combined formulation or coordinate treatment method that is combinatorially effective or coordinately useful to treat autoimmune disorders and/or one or more symptom(s) of an autoimmune disorder or condition in the subject. Exemplary combinatorial formulations and coordinate treatment methods in this context employ a membrane estrogen receptor agonist in combination with one or more secondary or adjunctive therapeutic agents selected from, e.g., immunoglobulins (e.g., a CTLA4Ig, such as BMS-188667; see, e.g., Srinivas et al., J. Pharm. Sci. 85(1):1-4, (1996), incorporated herein by reference); copolymer 1, copolymer 1-related peptides, and T-cells treated with copolymer 1 or copolymer 1-related peptides (see, e.g., U.S. Pat. No. 6,844,314, incorporated herein by reference); blocking monoclonal antibodies, transforming growth factor-β, anti-TNF α antibodies; or steroidal agents.

In certain embodiments the invention provides combinatorial autoimmune disease ameliorating formulations comprising a membrane estrogen receptor agonist and one or more adjunctive agent(s). Within such combinatorial formulations. membrane estrogen receptor agonists and the adjunctive agent(s) will be present in a combined formulation in autoimmune treating effective amounts, alone or in combination. In exemplary embodiments, membrane estrogen receptor agonists and another agent(s) will each be present in an autoimmune disorder treating amount (i.e., in singular dosage which will alone elicit a detectable anti-cellular proliferative response in the subject). Alternatively, the combinatorial formulation may comprise one or both of the membrane estrogen receptor agonist and non-membrane estrogen receptor agonists in sub-therapeutic singular dosage amount(s), wherein the combinatorial formulation comprising both agents features a combined dosage of both agents that is collectively effective in eliciting an autoimmune disorder treating response. Thus, one or both of the membrane estrogen receptor agonist and non-membrane estrogen receptor agonists may be present in the formulation, or administered in a coordinate administration protocol, at a sub-therapeutic dose, but collectively in the formulation or method they elicit a detectable autoimmune treating effective response in the subject.

To practice coordinate administration methods of the invention, a membrane estrogen receptor agonist may be administered, simultaneously or sequentially, in a coordinate treatment protocol with one or more of the secondary or adjunctive therapeutic agents contemplated herein. Thus, in certain embodiments a compound is administered coordinately with a non-membrane estrogen receptor agonist, or any other secondary or adjunctive therapeutic agent contemplated herein, using separate formulations or a combinatorial formulation as described above (i.e., comprising both a membrane receptor agonist, and a non-membrane receptor agonist). This coordinate administration may be done simultaneously or sequentially in either order, and there may be a time period while only one or both (or all) active therapeutic agents individually and/or collectively exert their biological activities. A distinguishing aspect of all such coordinate treatment methods is that the membrane estrogen receptor agonist exerts at least some autoimmune treating activity, which yields a favorable clinical response in conjunction with a complementary, or distinct, clinical response provided by the secondary or adjunctive therapeutic agent. Often, the coordinate administration of the membrane estrogen receptor agonist compound with the secondary or adjunctive therapeutic agent will yield improved therapeutic or prophylactic results in the subject beyond a therapeutic effect elicited by the membrane estrogen receptor agonist, or the secondary or adjunctive therapeutic agent administered alone. This qualification contemplates both direct effects, as well as indirect effects.

Within exemplary embodiments, a membrane estrogen receptor agonist will be coordinately administered (simultaneously or sequentially, in combined or separate formulation(s)), with one or more secondary agents, or other indicated therapeutic agents, e.g., selected from, for example, immunoglobulins (e.g., a CTLA4Ig, such as BMS-188667; see, e.g., Srinivas et al., J. Pharm. Sci. 85(1):1-4, (1996), incorporated herein by reference); copolymer 1, copolymer 1-related peptides, and T-cells treated with copolymer 1 or copolymer 1-related peptides (see, e.g., U.S. Pat. No. 6,844,314, incorporated herein by reference); blocking monoclonal antibodies, transforming growth factor-β, anti-TNF α antibodies; steroidal agents.

As noted above, in all of the various embodiments of the invention contemplated herein, the methods and formulations may employ a membrane estrogen receptor agonist. In exemplary embodiments of the invention, G-1 is employed within the therapeutic formulations and methods for illustrative purposes.

The pharmaceutical compositions of the present invention may be administered by any means that achieve their intended therapeutic or prophylactic purpose. Suitable routes of administration for the compositions of the invention include, but are not limited to, oral, buccal, nasal, aerosol, topical, transdermal, mucosal, injectable, slow release, controlled release, iontophoresis, sonophoresis, and including all other conventional delivery routes, devices and methods. Injectable methods include, but are not limited to, intravenous, intramuscular, intraperitoneal, intraspinal, intrathecal, intracerebroventricular, intraarterial, subcutaneous and intranasal routes.

The compositions of the present invention may further include a pharmaceutically acceptable carrier appropriate for the particular mode of administration being employed. Dosage forms of the compositions of the present invention include excipients recognized in the art of pharmaceutical compounding as being suitable for the preparation of dosage units as discussed above.

Such excipients include, without intended limitation, binders, fillers, lubricants, emulsifiers, suspending agents, sweeteners, flavorings, preservatives, buffers, wetting agents, disintegrants, effervescent agents and other conventional excipients and additives. The pharmaceutical composition may additionally contain other pharmaceutically acceptable components, such a buffers, surfactants, antioxidants, viscosity modifying agents, preservatives and the like. Each of these components is well-known in the art. See, e.g., U.S. Pat. No. 5,985,310, the disclosure of which is herein incorporated by reference in its entirety. Other components suitable for use in the formulations of the present invention can be found in Remington's Pharmaceutical Sciences, Mace Publishing Company, Philadelphia, Pa., 17th ed. (1985). By way of illustration, the membrane estrogen receptor agonist can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1 to about 90% by weight of the active compound, and more generally from about 1 to about 30% by weight of the active compound.

If desired, the compositions of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps or other biocompatible matrices such as cholesterol.

Compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants.

Additional compositions of the invention can be prepared and administered in any of a variety of inhalation or nasal delivery forms known in the art. Devices capable of depositing aerosolized purified membrane estrogen receptor agonist formulations in the sinus cavity or pulmonary alveoli of a patient include metered dose inhalers, nebulizers, dry powder generators, sprayers, and the like. Methods and compositions suitable for pulmonary delivery of drugs for systemic effect are well known in the art. Additional possible methods of delivery include deep lung delivery by inhalation. Suitable formulations, wherein the carrier is a liquid, for administration, as for example, a nasal spray or as nasal drops, may include aqueous or oily solutions of membrane estrogen receptor agonist compositions and any additional active or inactive ingredient(s).

Further compositions and methods of the invention provide for topical administration of a membrane estrogen receptor agonist. Topical compositions may comprise a membrane estrogen receptor agonist along with one or more additional active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise an membrane estrogen receptor agonist compound dissolved or dispersed in a portion of a water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example, 24 hours. Transdermal delivery may also be enhanced through techniques such as sonophoresis.

Additional compositions and methods of the invention provide for liquid compositions for use as membrane estrogen receptor agonists. A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, or flavoring or coloring agent. Alternatively, a liquid formulation can be prepared from a reconstitutable powder. For example, a powder containing active compound, suspending agent, sucrose and a sweetener can be reconstituted with water to form a suspension; and syrup can be prepared from a powder containing active ingredient, sucrose and a sweetener.

Yet additional membrane estrogen receptor agonists of the invention are designed for parenteral administration, e.g. to be administered intravenously, intramuscularly, subcutaneously or intraperitoneally, including aqueous and non-aqueous sterile injectable solutions which, like many other contemplated compositions of the invention, may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Additional compositions and formulations of the invention may include polymers for extended release following parenteral administration. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

A typical composition for intramuscular or intrathecal administration will be of a suspension or solution of active ingredient in an oil, for example, arachis oil or sesame oil. A typical composition for intravenous or intrathecal administration will be a sterile isotonic aqueous solution containing, for example, active ingredient and dextrose or sodium chloride, or a mixture of dextrose and sodium chloride. Other examples are lactated Ringer's injection, lactated Ringer's plus dextrose injection, Normosol-M and dextrose, Isolyte E, acylated Ringer's injection, and the like. Optionally, a co-solvent, for example, polyethylene glycol, a chelating agent, for example, ethylenediamine tetracetic acid, and an anti-oxidant, for example, sodium metabisulphite may be included in the formulation. Alternatively, the solution can be freeze dried and then reconstituted with a suitable solvent just prior to administration.

In more detailed embodiments, compositions of the invention may comprise a membrane estrogen receptor agonist compound encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules., respectively; in colloidal drug delivery systems (for example, Liposomes, albumin microspheres, microemulsions, nano-particles and nanocapsules); or within macroemulsions.

The invention disclosed herein will also be understood to encompass methods and compositions comprising membrane estrogen receptor agonists, using in vivo metabolic products of the said compounds (either generated in vivo after administration of the subject precursor compound, or directly administered in the form of the metabolic product itself). Such products may result for example from the oxidation, reduction, hydrolysis, amidation, esterification and the like of the administered compound, primarily due to enzymatic processes. Accordingly, the invention includes methods and compositions of the invention employing compounds produced by a process comprising contacting a membrane estrogen receptor agonist with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

Also provided are kits and systems that find use in practicing the subject methods, as described above. For example, kits and systems for practicing the subject methods may include one or more pharmaceutical formulations, which include one or more membrane estrogen receptor agonist and/or another therapeutic agent. As such, in certain embodiments the kits may include a single pharmaceutical composition, present as one or more unit dosages, where the composition includes both the membrane estrogen receptor agonist, and possibly another therapeutic agent. In yet other embodiments, the kits may include three or more separate pharmaceutical compositions, each containing a membrane estrogen receptor agonist and one or more additional therapeutic agents, or a combination of these elements.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert., etc. Yet another means would be a computer readable medium, e.g., diskette, CD, etc., on which the information has been recorded. Yet another means that may be present is a website address which may be used via the Internet to access the information at a removed site. Any convenient means may be present in the kits. For example, a kit according to one embodiment includes as a first component (a) instructions for using a membrane estrogen receptor agonist agent.

As used herein and in the appended claims, the singular forms “a,” “an,” and “the” include plural reference unless the context clearly dictates otherwise. The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

The terms and expressions that have been employed are used as terms of description arid not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The following examples illustrate certain aspects of the invention, but are not intended to limit in any manner the scope of the invention.

### EXAMPLES

The results presented below demonstrate that the membrane: estrogen receptor (mER), GPR30, is both necessary and sufficient for full 17β estradiol (E2) mediated protection against experimental autoimmune encephalomyelitis (EAE). Moreover, the agonist G-1 which selectively activates GPR30 without engagement of intracellular estrogen receptors (iERs), retained E2's ability to protect against clinical and histological EAE without obvious estrogenic side effects.

### Example 1

### Generation of GPR30-Mice

C57BL/6 (B6) mice were purchased from the animal service at National Cancer Institute (Frederick, Md.). Animals were housed and cared for according to institutional guidelines in the animal resource facility at the Veterans Affairs Medical Center (Portland, Oreg.).

Gper, the gene encoding GPR30, was targeted in 129 SvEvTac embryonic stem (ES) cells by specific vector with Neo insertion. The GPR30KO construct was based on BAC RP23-276 B20 from the RPCI-23 female C57BL/6 mouse library, chromosome #5. The BAC was digested with SacI (as well as EcoRV and Avill) and cloned into pBluescript II SK (pBSIISK) plasmid. Following Southern colony hybridization, two clones were identified to have the 5′ and the 3′ ends of Gper gene. Two intermediate molecules were then made by subcloning into pBSTISK plasmid: one that reconstructed a BamHI-KpnI fragment (site #1), and another into which a KpnI (site #1) to KpnI (site #2) fragment was inserted. All DNA was extensively sequenced to confirm the in silico database. The targeting vector was then constructed as following: 1) a 1.2 kb fragment from the EcoRI site to the second KpnI site (short arm of targeting construct) of mouse GPR30 was cloned into the plasmid pSPORT1 (EcoRI/Kpn I); 2) a 4 kb fragment from BamHI to XhoI (long arm of construct) of Gper was then cloned into the BamHI/SalI sites of above intermediate; 3) a blunt ended XhoI/SalI 1.2 kb fragment of pMCNeoPolyA containing the TK promoter, the Neo gene and associated polyA sequence was finally cloned into the SmaI site just 5′ of the Gper short arm and 3′ of long arm. All cloning junctions were sequenced as a check, and sequencing confirmed the orientation of Neo^{r} cassette. Initial ES cell screening was conducted using the 3′ probe, which was subcloned as a 1067 by KpnI/ApaI fragment into KpnI/ApaI of pGEM7. Restriction digestion with Hind III yields a 6.9 kb band in the wild type (WT) allele, and a 4.7 kb band in the targeted (Tgt) allele. Over 300 ES cell clones were screened and a single (+) clone containing both expected bands, denoted T142, was isolated. This clone was subsequently reanalyzed using both probes. The 5′ probe was subcloned as a 1004 by Ns I/StuI fragment into PstI/EcoRV of pBlueScript II. Restriction digestion with EcoRI yielded a 10.6 kb band in the WT and 8.2 kb in the Tgt allele, as expected. Restriction digestion with either Sad or BamHI and use of the 3′ probe respectively yielded bands of either 7.7 kb or 6.2 kb for the WT allele, and 5.5 kb or 8.2 kb for the Tgt allele, all as expected. The targeted ES cells were microinjected into C57BL/6 blastocysts, which were then implanted into recipient pseudo-pregnant CD1 female mice. Chimeric male mice with high ES cell contribution were then backcrossed to C57BL/6 females germ line transmission was identified by coat color, and then confirmed by genotyping PCR and southern blot conducted using standard alkaline transfer after agarose gelelectrophoresis onto GeneScreen neutral nylon membrane (PerkinElmer, Waltham, Mass.). All probes were labeled with [α-32P]-dCTP using the Prime-It random primer labeling kit (Stratagene, La Jolla, Calif.), and blots were visualized by autoradiography. Female homozygous GPR30 deficient mice (N2) offspring were backcrossed with wildtype C57BL/6J males from Jackson Laboratory (Bar Harbor, Me.). Their heterozygous progeny (N3) were bred to produce mixed genotypes, and the homozygotic GPR30-deficient animals were selected for breeding a fourth (N4) generation of homozygous Gper−/. Mice homozygous for GPR30KO are viable, fertile, and do not display any gross physical, immunological, reproductive, or neurological abnormalities. GPR30KO and the WT control mice used in this experiment were backcrossed with C57BL/6J mice for 6 times before breeding with homozygous breeders. (Wang et al. Mol. Endocrinology, 22 (3): 636-648 (2008)).

### Example H

### Contribution of GPR30 to E2-Induced Protection Against EAE

Age matched WT C57BL/6 female mice purchased from the animal service at National Cancer Institute (Frederick., MD), GPR30KO and heterozygous (GPR30^{+/-}) female mice prepared as described in Example 1 from the same colony were implanted with 2.5 mg/60 day release 17β estradiol (E2) (Innovative Research of America, Sarasota, Fla.)) or placebo pellets one week prior to immunization with mouse (m)MOG-35-55 peptide (MEVGWYRSPFSRVVHLYRNGK SEQ ID NO:1) in complete Freund's adjuvant (CFA) with additional pertussis toxin (PTX, List Biological Laboratories, Campbell, Calif.) on days 0 and 2. The (m)Mog-35-55 peptide was synthesized using solid phase techniques and purified by HPLC (Beckman Institute, Stanford University, Palo Alto, Calif.).

Experimental autoimmune encephalornyelitis (EAE) was induced by inoculating the mice s.c. in the flanks with 0.2 ml of an emulsion containing 200 μg of mMOG-35-55 peptide and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2-moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6-paraplegia with severe forelimb weakness or severe ataxia, moribund condition or dead. The mice were scored for EAE development and euthanized 25 days after immunization. The experiment was repeated 3 times with at least 7 mice in each group.

Serum levels of E2 were determined by radioimmunoassay (RIA) after Sephadex LH-20 column chromatography (GE Healthcare Life Sciences, Piscataway, N.J.). Representative animals were bled by cardiac puncture, and the blood was allowed to clot at 4° C. overnight. The samples were centrifuged, and the sera were collected and stored at −80° C. until hormone analysis was performed. All samples were analyzed in a single assay for each hormone.

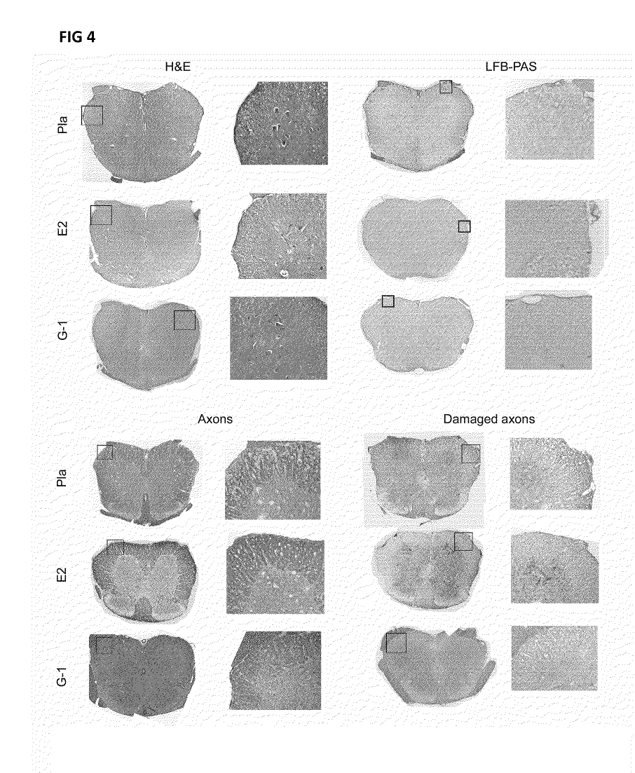

As shown in FIG. 3, serum E2 levels were 1.5-2 ng/ml. (Steroid levels were measured in sera collected from placebo, 3.7 mg G-1- and 2.5 mg E2 treated mice used in

| Genotype | pellet | n | Incidence | Mortality | Onset | Peak | Daily | CDI |
|---|---|---|---|---|---|---|---|---|
| WT | Placebo | 10 | 9/10 | 0/10 | 15.3 ± 2.1 | 4.1 ± 0.9 | 3.4 ± 0.7 | 33.1 ± 16.3 |
| | E2 | 10 | 0/10 | 0/10 | — | 0** | 0** | 0** |
| GPR30KO | Placebo | 7 | 6/7 | 0/7 | 13.4 ± 0.53 | 3.8 ± 1.7 | 3.6 ± 0.9 | 40.1 ± 13.2 |

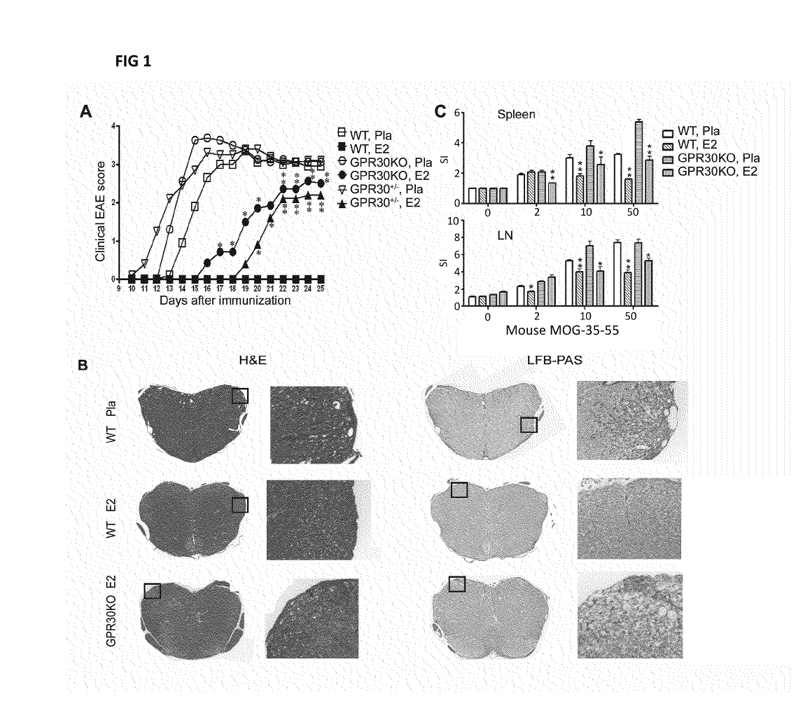

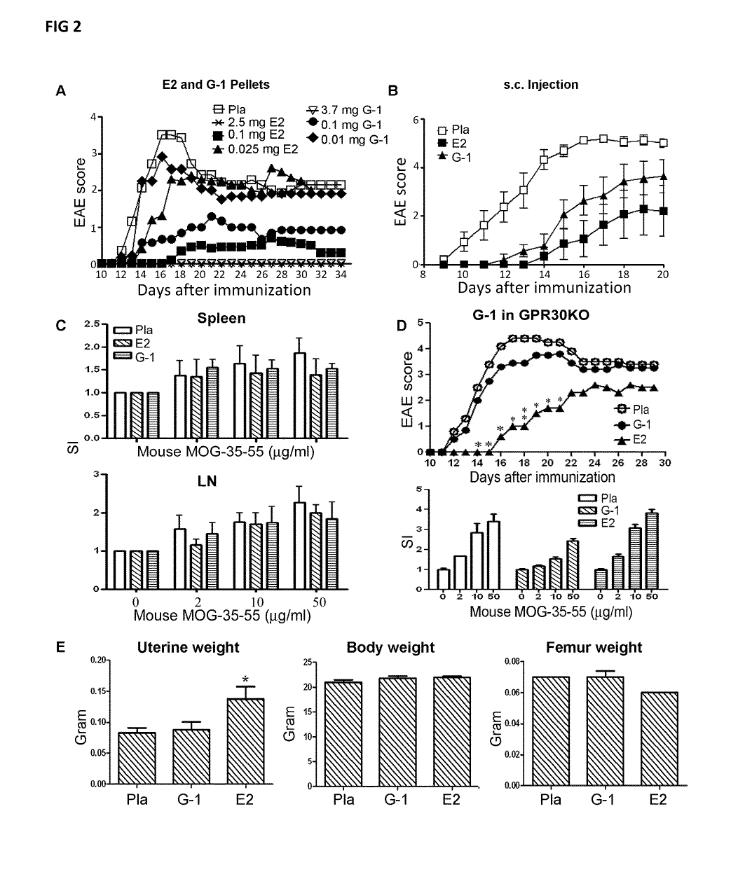

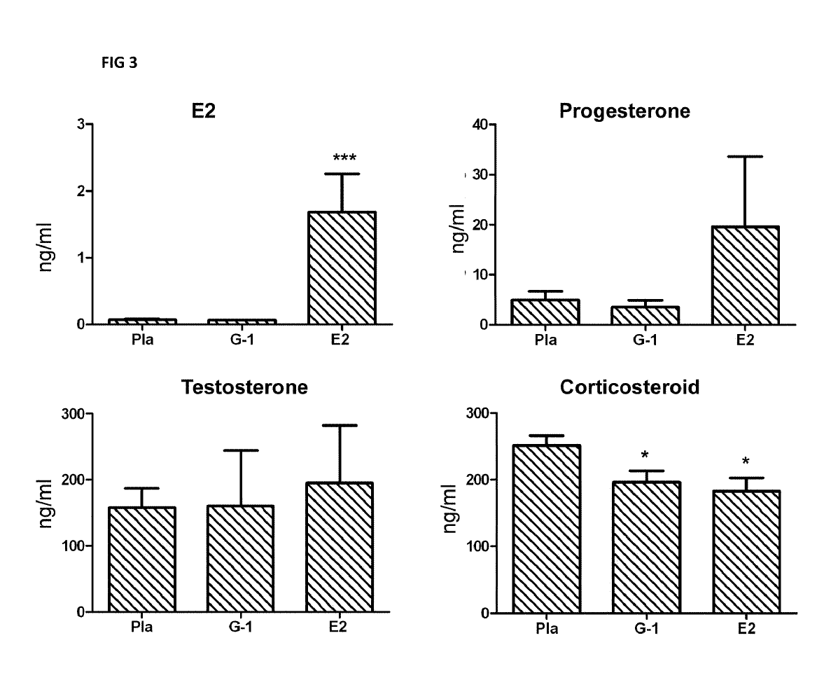

the clinical experiments shown in FIG. 2A. (***P<0.001 or *P<0.05). Placebo treated WT and GPR30KO mice developed severe EAE with onset on day 11 and peak about Day 15, whereas E2-treated WT mice developed no sign of the disease. In contrast, E2 treated GPR30KO and heterozygous mice had delayed onset and were only partially protected from EAE. (FIG. 1A and Table I)

TABLE I
Gene disruption of GPR30 weakens the therapeutic effects of E2 on EAE^{a}.

| | | | | | | | | |
|---|---|---|---|---|---|---|---|---|
| GPR30^{w/-} | E2 | 7 | 4/7 | 0/7 | 18.0 ± 1.4** | 2.5 ± 2.3^{#} | 3.7 ± 1.3 | 17.2 ± 17.4*^{#} |
| | Placebo | 10 | 9/10 | 0/10 | 13 ± 2.8 | 3.6 ± 1.6 | 3.5 ± 0.6 | 38.9 ± 31.4 |
| | E2 | 10 | 5/10 | 0/10 | 20.0 ± 1.0** | 2.1 ± 2.3*^{#} | 2.6 ± 0.8 | 11.3 ± 13.0**^{#} |

^{a}Mice were immunized one week after implantation with 2.5 mg/60 day release E2 pellets. The experiment was concluded 25 days after immunization.
*P < 0.05 or
**P < 0.01 as compared to placebo control,
^{#}P < 0.05 as compared to E2-treated WT.

Spinal cords were dissected out for histology. Intact spinal columns were removed and dissected after fixation in 4% paraformaldehyde, dehydrated and embedding in paraffin. To examine neuroinflammation, the sections were stained with hematoxylin and eosin (H&E). To examine demyelination, the sections were stained with Luxon fast blue plus periodic acid Schiff (LFB-PAS).

Pathologically, all of the placebo-treated mice had substantial immune cell infiltration and demyelination in the central nervous system (FIG. 1B) whereas no pathological signs of EAE were found in E2-treated WT mice. In E2-treated GPR30-KO mice, however, cellular infiltration and demyelination were visibly present, but at a reduced level. T-cell responses to MOG peptide were similarly reduced by E2 treatment in both WT and GPR30Ko mice (FIG. 1C (*P<0.05 or **P<0.01 as compared to placebo control, ^{#}P<0.05 as compared to E2-treated WT)) suggesting that GPR30 is not directly linked to suppression of T cell proliferation. Taken together, these results indicate a significant, but not exclusive role for GPR30 in E2-mediated protection against EAE.

### Example III

### Efficacy of G-1 and E2 in Protecting Wild-Type C57BL/6 Mice Against EAE

C57BL/6 (B6) mice were purchased from the animal service at National Cancer Institute (Frederick, Md.). Animals were housed and cared for according to institutional guidelines in the animal resource facility at the Veterans Affairs Medical Center (Portland, Oreg.).

1.8, 0.1 or 0.01 mg/40 day release pellets of G-1 (Cayman Chemicals, Ann Arbor, Mich., Innovative Research of America, Sarasota, Fla.), 2.5, 0.1 or 0.025 mg/60 day release pellets of 17β estradiol (E2) (Innovative Research of America, Sarasota, Fla.) or placebo (Innovative research of America) were administered to mice 7 days prior to induction of experimental autoimmune encephalomyelitis (EAE). EAE was induced by inoculating the mice s.c. in the flanks with 0.2 ml of an emulsion containing 200 μg of mMOG-35-55 peptide (Beckman Institute, Stanford University, Palo Alto, Calif.) and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. The mice were scored for EAE development and euthanized 34 days after immunization for ex vivo studies. The experiment was repeated 2 times with 5-8 mice in each group.

As shown in FIG. 2A and Table II, treatment with G-1 delayed and ameliorated EAE in a dose-dependent manner. Administration of either 1.8 mg/40 day release G-1 (Cayman Chemicals, Ann Arbor, Mich., Innovative Research of America, Sarasota, Fla.) or the molar equivalent level of 2.5 mg/60 day release E2 (Innovative Research of America, Sarasota, Fla.) pellets completely protected the mice from clinical EAE, with lower doses of G-1 being less effective than E2.

TABLE II
The protective effects of G-1 vs. E2 in wild-type B6 mice^{b}.

| Treatment | n | Incidence | Mortality | Onset | Peak | Daily | CDI |
|---|---|---|---|---|---|---|---|
| Placebo pellets | 7 | 7/7 | 0/7 | 13.7 ± 1.1 | 3.9 ± 0.3 | 2.0 ± 0.54 | 51.9 ± 13.5 |
| 1.8 mg G-1 | 7 | 0/7** | 0/7 | — | 0** | 0** | 0** |
| 0.1 mg G-1 | 6 | 3/6 | 0/6 | 16.7 ± 2.3* | 1.5 ± 1.6** | 0.75 ± 1.3* | 18.8 ± 28.3* |
| 0.01 mg G-1 | 6 | 5/6 | 0/6 | 13.6 ± 1.6 | 3.1 ± 1.7 | 1.8 ± 1.6 | 43.8 ± 39.9 |
| 2.5 mg E2 | 7 | 0/7** | 0/7 | — | 0** | 0** | 0** |
| 0.1 mg E2 | 6 | 3/10** | 0/10 | 21.0 ± 6.1** | 0.8 ± 1.2** | 0.3 ± 0.6** | 9.1 ± 14.8** |
| 0.025 mg E2 | 5 | 4/5 | 0/5 | 14.8 ± 0.5 | 2.8 ± 1.6 | 1.7 ± 1.0 | 37.0 ± 21.6 |
| Placebo injection | 8 | 8/8 | 0/7 | 11.1 ± 2.0 | 5.3 ± 2.4 | 5.9 ± 2.4 | 42.7 ± 8.1 |
| G-1 injection | 7 | 6/8 | 1/8 | 13.7 ± 1.3 | 3.7 ± 1.6* | 2.7 ± 1.3* | 19.7 ± 11.3* |
| E2 injection | 7 | 3/8* | 1/8 | 15.3 ± 1.5 | 2.3 ± 2.9* | 2.3 ± 0.3* | 10.5 ± 13.9** |

^{b}Mice were immunized one week after implantation with G-1 or E2 pellets at different doses. The experiment was concluded 34 days after immunization.
*P < 0.05 or
**P < 0.01 as compared to placebo controls.

In a separate experiment, mice were injected daily s.c. underneath the neck skin with G-1 (20 μg/mouse/day in 100 μl of 10% ethanol and 90% olive oil) (Cayman Chemicals, Ann Arbor, Mich.), E2 (1 μg/mouse/day in 100 μl of 10% ethanol and 90% olive oil) (Sigma-Aldrich, St. Louis, Mo.) or placebo (100 μl of 10% ethanol and 90% olive oil) one week prior to induction of EAE. EAE was induced by inoculating the mice s.c. in the flanks with 0.2 ml of an emulsion containing 200 μg of mMOG-35-55 peptide (Beckman Institute, Stanford University, Palo Alto, Calif.) and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. The mice were monitored daily for changes in clinical EAE scores and were euthanized 20 days after immunization (P<0.05 or 0.01 for E2-treatment groups from Day 11 to 20, and for G-1 treatment groups from Day 11 to 17 as indicated by One-way Anova followed by Newman-Kuels multiple comparisons test.) As shown in FIG. 2B and Table II, the injections were partially protective against EAE.

Splenocytes and lymph node cells were harvested ex-vivo from G-1 and E2 implanted mice and cultured in a 96-well flat bottom tissue culture plate at 4×10⁵ cells/well in stimulation medium in the presence of APC, irradiated (2500 rad) syngenic thymocytes at a ration of 1:10 (T:APCs) wither with or without mMog 35-55 peptide at varying concentrations. The cells were incubated for 3 days at 37° C. in 7% CO₂ and pulsed with 0.5 μCi of [³H] thymidine (Perkin-Elmer, Boston, Mass.) for the final 18 h of incubation. The cells were harvested onto glass fiber filters, and incorporated radioactivity was measured by a liquid scintillation counter. The cpm values (mean±SD) were calculated from triplicate wells. Stimulation index (SI) was calculated by dividing the experimental cpm by the control cpm. As shown in FIG. 2C, neither G-1 nor E2 treatment significantly altered ex vivo T cell proliferation responses to mMog-35-55 peptide.

### Example IV

### Requirement of GPR30 for G-1 Induced Protection Against EAE

GPR30KO mice were prepared as in Example I. C57BL/6 (B6) mice were purchased from the animal service at National Cancer Institute (Frederick, Md.). Animals were housed and cared for according to institutional guidelines in the animal resource facility at the Veterans Affairs Medical Center (Portland, Oreg.).

GPR30 mice were implanted with placebo, 1.8 mg/40 day release G-1 (Cayman Chemicals, Arm Arbor, Mich. and Innovative Research of America, Sarasota, Fla.), 2.5 mg/60 day release 17β estradiol (E2) (Innovative Research of America, Sarasota, Fla.) pellets, or placebo 7 days prior to immunization. Experimental autoimmune encephalomyelitis (EAE) was induced by inoculating the mice s.c. in the flanks with 0.2 ml of an emulsion containing 200 μg of mMOG-35-55 (Beckman Institute, Stanford, Palo Alto, Calif.) peptide and an equal volume of complete Freund's adjuvant (CFA) containing 200 μg of heat killed Mycobacterium tuberculosis 1437RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. The experiment was concluded 29 days after immunization for ex vivo experiments and was repeated 2 times with 7-10 mice in each group.

Although the protective effect of E2 against EAE was only partially offset by the absence of GPR30, treatment with G-1 was completely ineffective in GPR30KO mice (FIG. 2D (*P<0.05 or **P<0.01 compared to placebo control) and Table III)
Table III The protective effect of G-1 was abrogated in GPR30KO mice^{c}.

^{c}GPR30KO mice were immunized one week after implantation with 1.8 mg/40 day release G-1 or 2.5 mg/60 day release E2 pellets for a week. The experiment was concluded 29 days after immunization. *P<0.05 or **P<0.01 as compared to placebo control.

Neither G-1 nor E2 affected T cell proliferation to mMog-35-55.

Treatment with G-1 in viva lacked the “estrogenic effects” of E2. In contrast to E2, G-1 treatment did not significantly change the weight of uteri, a prominent and well-known estrogenic effect (FIG. 2E (*P<0.05 compared to placebo control)). Additionally, there were no abnormalities by hematoxylin and eosin stain (H&E) staining in the liver, eyes, heart, mammary gland, brain, spleen, kidney, muscle or lung from 1.8 mg G-1 treated naive mice.

To rule out the possibility that G-1 prevented EAE by regulating endogenous steroid hormones, levels of E2, progesterone and corticosteroid were measured in sera from mice treated with placebo, 3.7 mg G-1 or 2.5 mg E2. The results showed that G-1 slightly lowered the level of corticosteroid but did not affect any of the other steroid hormones tested (FIG. 3 (***P<0.001 or *P<0.05)), thus ruling out the possibility that

| Treatment | n | Incidence | Mortality | Onset | Peak | Daily | CDI |
|---|---|---|---|---|---|---|---|
| Placebo | 10 | 9/10 | 0/10 | 14.7 ± 1.5 | 4.0 ± 1.6 | 2.5 ± 1.1 | 49.7 ± 22.8 |
| G-1 | 8 | 8/8 | 0/8 | 13.8 ± 1.2 | 4.5 ± 0.7 | 3.0 ± 0.7 | 59.9 ± 13.4 |
| E2 | 7 | 4/7 | 0/7 | 18.0 ± 1.4** | 2.5 ± 2.3^{#} | 3.7 ± 1.3 | 17.2 ± 17.4*^{#} |

upregulation of endogenous anti-inflammatory steroid hormones was responsible for the clinical improvement caused by G-1 treatment.

The mice from the clinical experiment shown in FIG. 2A were euthanized at the end of the experiment and spinal cords were dissected out for histology. Intact spinal cords were removed and dissected after fixation in 4% paraformaldehyde, dehydrated and embedding in paraffin. To examine neuroinflammation, the sections were stained with hematoxylin and eosin (H&E). To examine demyelination, the sections were stained with Luxon fast blue plus periodic acid Schiff (LFB-PAS).

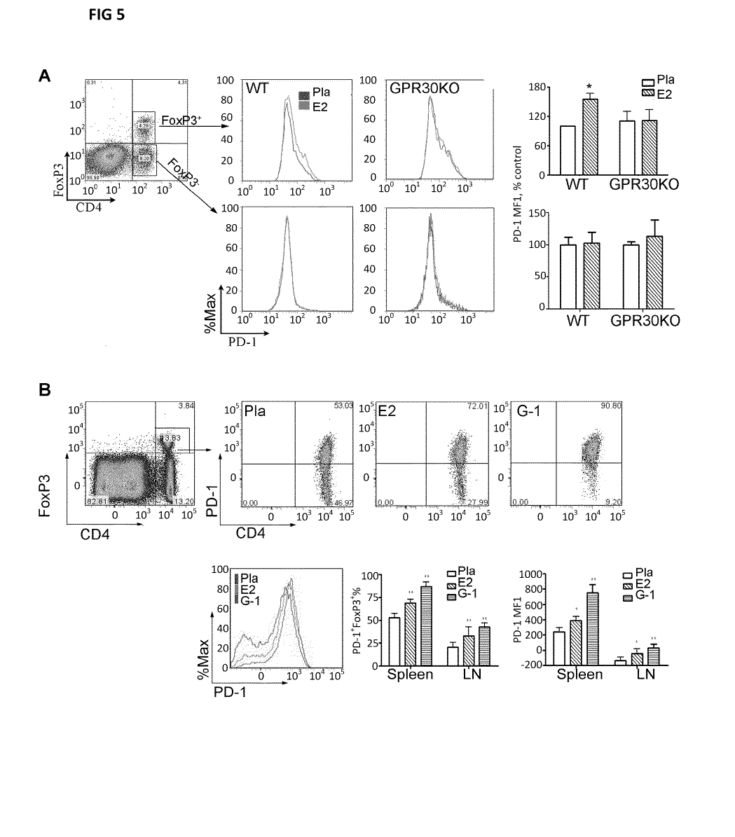

Consistent with the clinical observations, both G-1 and E2 treatments markedly ameliorated immune cell infiltration and demyelination in the CNS as indicated by H&E staining and LFB-PAS staining (FIG. 4, upper panels). Moreover, both agents reduced the level of axonal damage in spinal cord sections (FIG. 4, lower panels). Existing axons can be visualized by immunohistochemical staining with SMI312 (Covance, Princeton, N.J.), an antibody cocktail that stains phosphrylated neurofilaments (NFL). The degree of ongoing damage can be seen by staining non-phosphorylated bneurofilaments (NPNFL) with SMI312, which specifically detects injured and demyelinated axons.

To visualize axonal damage, spinal cords were fixed in 4% paraformaldehyde (maass/volume in PBS, pH 7.4) at 4° C. for at least 48 h. The spinal cords were dissected out from the columns, cut into sections 1-2 mm in length from the sampled thoracic or limbic cords, dehydrated and embedded in paraffin blocks. Then, 10 μm thick sections were cut from paraffin blocks and mounted onto pre-cleaned microscope slides. The sections were dewaxed and rehydrated sequentially by xylene (2 min), gradient ethanol (100%, 95%, 85%, 2 min each) and PBS (5 min) and then cooked (120° C.) in antigen unmasking agent Trilogy® (Cell Marque, Hot Springs, Ark.) for 10 min in a pressure steamer. The endogenous peroxidase activity was blocked with 3% hydrogen peroxide in tap water for 5 min. the sections were incubated 1 h in a working solution of Mouse IgG blocking reagent from the Vector® M.O.M.™ Immunodetection peroxidase kit (Vector Laboratories, Burlingame, Calif.), and then incubated sequentially with primary antibody (SMI312 1:3000 or SMI32 1:1000 diluted in M.O.M™ dilutents) for 30 min, M.O.M™ biotinylated anti-mouse IgG reagent for 10 min, VECTASTAIN® ABC reagent for 5 min, arid DakoCytomation liquid DAB substrate (DakoCytomation, Carpinteria, Calif.) until sections turned light brown. The slides were counterstained with hematoxylin for 30-60 s to visualize nuclei, washed with tap water, dehydrated, and mounted with Cytoseal™ XYL mounting medium (Richard-Allan Scientific, Kalamazoo, Mich.). The sections were analyzed by light microscopy after staining and recorded with a digital camera.

In placebo treated WT mice, axonal staining was markedly reduced in the presence of inflammatory mononuclear cells, resulting in severe loss of SMI312 staining in the outer region of white matter where most neuroinflammation occurred (FIG. 4, lower left panel.) In contrast, axons in the spinal cords of G-1 and E2 treated mice were well preserved. Additionally, sections from both G-1 and E2 treated mice showed much less SMI32 staining in the white matter of the spinal cords (FIG. 4, lower right panel). G1 and E2 therefore induced comparable neuroprotective effects in EAE.

### Example V

### Upregulation of Foxp2 or PD-1 Epression in Treg Cells

Age matched WT C57BL/6 from the animal service at National Cancer Institute (Frederick, Md.), GPR30KO prepared as in Example 1, or PDD-KO mice (Dr. Tasuku Honjo at Hyoto University, Kyoto, Japan backcrossed with B6 mice for more than 10 generations) female mice from the same colony were implanted with 2.5 mg/60 day release 17β estradiol (E2) (Innovative Research of America, Sarasota, Fla.) or placebo pellets one week prior to immunization with mouse (m)MOG-35-55 peptide (MEVGWYRSPFSRVVHLYRNGK SEQ ID NO:1) in complete Freund's adjuvant (CFA) with additional pertussis toxin (PTX, List Biological Laboratories, Campbell, Calif.) on days 0 and 2. The (m)Mog-35-55 peptide was synthesized using solid phase techniques and purified by HPLC (Beckman Institute, Stanford University, Palo Alto, Calif.).

Experimental autoimmune encephalomyelitis (EAE) was induced by inoculating the mice s.c. in the flanks with 0.2 ml of an emulsion containing 200 μg of mMOG-35-55 peptide and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. The mice were scored for EAE development and euthanized 25 days after immunization.

Splenocytes or lymph node cells from GPR30KO mice were obtained at the end of the clinical experiment in FIG. 1A and stained for FoxP3 and PD-1, as well as cellular markers for T cells and antigen presenting cells (APC). For membrane staining, 1 million cells were stained at 4° C. in the dark with appropriate Ab dilutions in staining buffer (PBS containing 0.5% BSA and 0.02% sodium azide), Intracellular staining for FoxP3 was performed following the protocol recommended by eBioscience (San Diego, Calif.). Briefly, 4 million cells were surface stained following standard procedures. After washing, the cells were fixed overnight and washed twice with 0.5 ml of permeabilization buffer. The cells were co-stained for 15 min with FcBlock (Becton Dickinson, Franklin Lake, N.J.) and PerCp (eBioscience, San Diego, Calif.) followed 30 min later with fluorescent-labeled antibodies to FoxP3 (eBioscience, San Diego, Calif.) or isotype control. The cells were then washed twice with 2 ml of permeabilization buffer and once with 1 ml of staining buffer, and re-suspended in staining buffer. Flow cytometry data were collected on LSRII and FACSCalibur flow cytometers (BD Bioscience, Franklin Lakes, N.J.), and analyzed using FlowJo software (Tree Star, Ashland, Oreg.). Data represent 50,000-100,000 events unless otherwise noted.

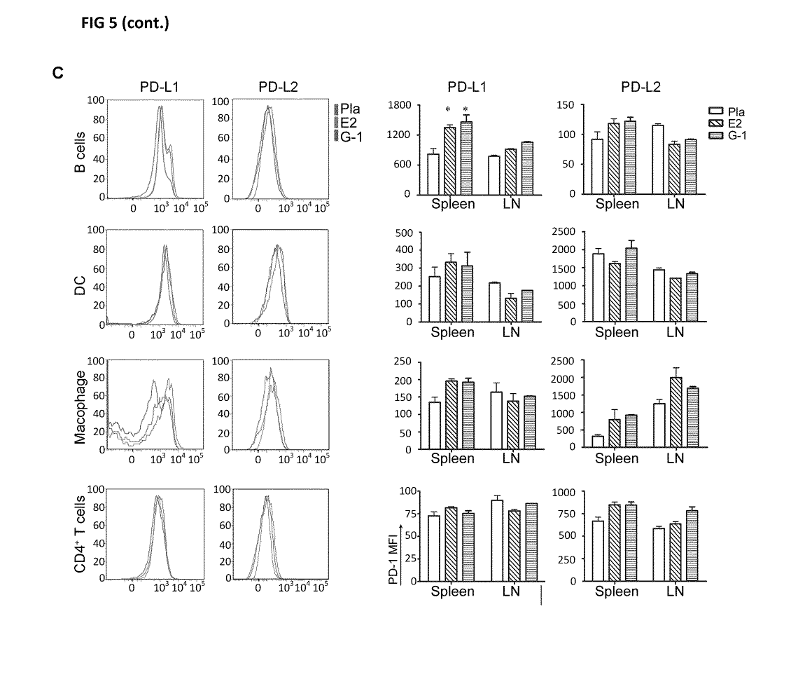

The expression levels of PD-1 were analyzed by gating on CD4⁺FoxP3⁺ or CD4⁺FoxP3⁻ T cells from WT or GPR30 mice. The bar charts in FIG. 5A show the average mean fluorescence intensity (MFI) of Pd-1 in CD4⁺FoxP3⁺ or CD4⁺FoxP3⁻ from all mice in each group. The results show that GPR30 was not required for E2 induced upregulation of CD4⁺FoxP3⁻ T cells. However, E2 induced upregulation of PD-1 expression in these cells was abolished in splenocytes from PD-1KO mice (FIG. 5A). E2 did not induce a significant shift in PD-1 expression in the non-regulatory CD4⁺Fox P3⁻ T cells in WT or PD-1KO mice. GPR30 is therefore required for E2-induced upregulation of PD-1 Treg cells.

### Example VI

### Effect of GRP30 on PD-1 in CD4⁺FoxP3⁺Treg Cells

Age matched WT C57BL/6 and GPR30KO female mice from the same colony were implanted with 2.5 mg/60 day release 17β estradiol (E2)(Innovative Research of America, Sarasota, Fla.), G-1 or placebo pellets one week prior to immunization with mouse (m)MOG-35-55 peptide (MEVGWYRSPFSRVVHLYRNGE: SEQ ID NO:1) in complete Freund's adjuvant (CFA) with additional pertussis toxin (PTX, List Biological Laboratories, Campbell, Calif.) on days 0 and 2. The (m)Mog-35-55 peptide was synthesized using solid phase techniques and purified by HPLC (Beckman Institute, Stanford University, Palo Alto, Calif.).

Experimental autoimmune encephalomyelitis (EAE) was induced by inoculating the mice s.c. in the flanks with 0.2 ml of an emulsion containing 200 μg of mMOG-35-55 peptide and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. The mice were scored for EAE development and euthanized 25 days after immunization. The experiment was repeated 2 times with a total of 7-10 mice per group.

Splenocytes or lymph node cells were harvested from placebo-, E2- or G-1 treated mice after EAE induction as shown in FIG. 2A and stained for CD4, FoxP3 and PD-1. The expression levels of Pd-1 were analyzed by gating on CD4⁺FoxP3⁺ cells from placebo-, E2, or G-1 treated WT mice. For membrane staining, 1 million cells were stained at 4° C. in the dark with appropriate Ab dilutions in staining buffer (PBS containing 0.5% BSA and 0.02% sodium azide), Intracellular staining for FoxP3 was performed following the protocol recommended by eBioscience. Briefly, 4 million cells were surface stained following standard procedures. After washing, the cells were fixed overnight and washed twice with 0.5 ml of permeabilization buffer. The cells were co-stained for 15 min with FcBlock (Becton Dickinson, Franklin Lake., NJ) and IgG PerCp (eBioscience, San Diego, Calif.) followed 30 min later with fluorescent-labeled antibodies to FoxP3 (eBioscience, San Diego, Calif.) or isotype control. The cells were then washed twice with 2 ml of permeabilization buffer and once with 1 ml of staining buffer, and re-suspended in staining buffer. Flow cytometry data were collected on LSRII and FACSCalibur flow cytometers (BD Bioscience, Franklin Lakes, N.J.), and analyzed using FlowJo software (Tree Star, Ashland, Oreg.). Data represent 50,000-100,000 events unless otherwise noted.

As seen in FIG. 5B, the dot plot shows the marked shift of CD4⁺FoxP3⁺ T cells from PD-1⁻ to PD-1⁺ after treatment with G-1 or E2. The histogram overlay compares the MFI of PD-1 in CD4⁺FoxP3⁺ T cells from different treatment groups. The bar charts show the average percentages of PD-1⁺ CD4⁺FoxP3⁺ T cells and the average MFI of PD-1 in CD4⁺FoxP3⁺ T cells from all mice in each grip[/*P<0.05 or **P<0.01 compared to placebo control.

Roughly half of the CD4⁺FoxP3⁺ cells from the spleen of placebo-treated mice were negative or low for PD-1 expression (FIG. 5B). Both E2 and G-1 treatments strongly enhanced the staining intensity of PD-1 and converted a majority of the CD4⁺FoxP3⁺PD-1⁻ cells to ⁺FoxP3⁺PD-1⁺ cells. Surprisingly, G-1 was even more potent than E2 in boosting PD-1 expression in CD4⁺FoxP3⁺ cells suggesting that the iER pathways for E2 might have opposing effects on GPR30.

### Example VII

### Suppressive Effect of GFP+PD-1⁺ Treg Cells in Comparison to GFP+PD-1⁻ Treg Cells

FoxP3-GFP “knock-in” mice (Dr. Steve Ziegler, Benaroya Research Institute, Seattle, Wash.) were immunized to induce EAE two weeks before suppression experiments.

Experimental autoimmune encephalomyelitis (EAE) was induced by inoculating the mice s.c. in the flanks with 0.2 ml of an emulsion containing 200 μg of mMOG-35-55 peptide and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE, according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead.

Single-cell suspensions of splenocytes were prepared by mincing the spleens through a vinyl screen. After the red blood cells were lysed, 2 ml cells were stained for CD4 and FoxP3 (intracellular staining). More than 98% of GFP⁺ cells were confirmed to be FoxP3⁺. The cells were stained with propidium iodide (PI) and antibodies for PD-1 and CD4. CD4⁺GFP⁺ cells were sorted into two distinct populations, PL)-1⁺ and PD-1⁻ cells using a FACSVantage (BD Immunocytornetry Systems, San Jose, Calif., USA). Two million CD4⁺GFP⁻ cells were sorted to act as responder cells. Splenocytes from a naive FoxP3-GFP+ mouse were depleted of T cells by magnetic sorting using CD90 Microbeads (Miltenyi Biotec, Bergisch Gladbach, Germany) and used as antigen-presenting cells (APCs). Briefly, cells were incubated on ice for 15 min with microbeads. After a wash, cells were sorted using the deplete program on an autoMACS magnetic cell sorter. The negative fraction was washed, re-suspended at 1×10⁶ per ml in RPMI containing 10% fetal bovine serum (FBS) and irradiated with 1800 rads in a cesium irradiator. The overall purity of T cell-depleted APCs was ˜0.80%. Suppression assays were performed in 96-well flat-bottomed plates (Becton Dickinson) in a final volume of 200 μl per well of RPMI containing 10% FBS. Both APCs and responder cells were plated at 0.5×10⁴ cells per well in triplicate and CD4⁺GFP⁺ suppressor cells were added at the various ratios of indicator:Tregs. Anti-CD3 antibody was added at a final concentration of 0.5 μg/ml. After 48 h, the plates were pulsed for 18 h with [³H]thymidine (Perkin-Elmer., Boston, Mass.), and the cells harvested on glass fiber filters and assessed for uptake of [³H]thymidine by liquid scintillation. The percent suppression was plotted versus indicator/suppressor cell ratios and a regression line was calculated. I₅₀ was determined as the ratio of indicator/suppressor cells that produced 50% suppression. Suppressive index (SI)=100−I₅₀.

Functional assays using the FoxP3-GFP “knock-in” mice indicated that GFP+PD-1⁺ Treg cells were more suppressive than GFP+PD-1⁻ Thus, activation of GPR30 may cause Treg cells to shift from PD-1⁻ to PD-1⁺ with enhanced suppressive function.

### Example VIII

### Effect of G-1 on PD-L1 and PD-L2

C57BL/6 (B6) mice were purchased from the animal service at National Cancer Institute (Frederick, Md.). Animals were housed and cared for according to institutional guidelines in the animal resource facility at the Veterans Affairs Medical Center (Portland, Oreg.).

1.8, 0.1 or 0.01 mg/40 day release pellets of G-1 (Cayman Chemicals, Ann Arbor, Mich., Innovative Research of America, Sarasota, Fla.), 2.5, 0.1 or 0.025 mg/60 day release pellets of 17β estradiol (E2) (Innovative Research of America, Sarasota, Fla.) or placebo (Innovative research of America) were administered to mice 7 days prior to induction of experimental autoimmune encephalomyelitis (EAE). EAE was induced by inoculating the mice s.c. in the flanks with 0.2 ml of an emulsion containing 200 μg of mMOG-35-55 peptide (Beckman Institute, Stanford University, Palo Alto, Calif.) and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. The mice were scored for EAE development and euthanized 34 days after immunization for ex vivo studies. The experiment was repeated 2 times with 5-8 mice in each group.

Splenocytes and lymph node cells were obtained at the end of the clinical experiment in FIG. 2A and stained for FoxP3 and PD-1 as well as cellular markers for T cells and APCs. For membrane staining, 1 million cells were stained at 4° C. in the dark with appropriate Ab dilutions in staining buffer (PBS containing 0.5% BSA and 0.02% sodium azide), Intracellular staining for FoxP3 was performed following the protocol recommended by eBioscience (San Diego, Calif.). Briefly, 4 million cells were surface stained following standard procedures. After washing, the cells were fixed overnight and washed twice with 0.5 ml of permeabilization buffer. The cells were co-stained for 15 min with FcBlock (Becton Dickinson, Franklin Lake, N.J.) and IgG PerCp (eBioscience, San Diego, Calif.) followed 30 min later with fluorescent-labeled antibodies to FoxP3 (eBioscience, San Diego, Calif.) or isotype control. The cells were then washed twice with 2 ml of permeabilization buffer and once with 1 ml of staining buffer, and re-suspended in staining buffer. Flow cytometry data were collected on LSRII and FACSCalibur flow cytometers (BD Bioscience, Franklin Lakes, N.J.), and analyzed using FlowJo software (Tree Star, Ashland, Oreg.). Data represent 50,000-100,000 events unless otherwise noted.

The inhibitory activity of PD-1 requires binding to co-stimulatory ligands including PD-L1 and PD-L2. As shown in FIG. 5C (*P<0.05 or **P<0.01 compared to placebo), both G-1 and E2 treatment upregulated PD-L1 expression on B cells and macrophages, but not dendritic cells or CD4⁺ cells, with essentially no effect on expression of PD-L2. Together with upregulation of PD-1, boosting PD-L1 expression by B cells and macrophages might be a contributing mechanism through which GPR30 mediates E2-induced protection against EAE.

### Example IX

### Evaluation of the Importance of G-1 Induced Upregulation of PD-1 in EAE Protection

PD-1KO (Dr. Tasuku Honjo, Kyoto Univeristy, Kyoto, Japan) mice were implanted with 2.5 mg/60 day release of 17β estradiol (E2) (Innovative Research of America, Sarasota, Fla.), 1.8 mg/40 day release G-1 (Cayman Chemicals, Aim Arbor, Mich. and Innovative Research of America, Sarasota, Fla.), or placebo pellets (Innovative Research of America, Sarasota, Fla.) one week prior to immunization to induce experimental autoimmune encephalomyelitis (EAE). EAE was induced by inoculating the mice s.c. in the flanks with 0.2 ml of an emulsion containing 200 μg of mMOG-35-55 peptide and an equal volume of complete Freund's adjuvant (CFA) containing 200 m of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. After 20 days, the mice were euthanized. The experiment was repeated 2 times with a total of 7-10 mice in each group.

| Treatment | n | Incidence | Mortality | Onset | Peak | Daily | CDI |
|---|---|---|---|---|---|---|---|
| Placebo | 5 | 5/5 | 1/5 | 12.2 ± 0.8 | 5.1 ± 0.2 | 3.5 ± 0.5 | 38.9 ± 5.1 |
| G-1 | 5 | 5/5 | 1/5 | 13.8 ± 1.3 | 4.8 ± 0.6 | 2.7 ± 0.8 | 30.6 ± 9.1 |
| E2 | 5 | 5/5 | 0/5 | 16.4 ± 0.9* | 4.1 ± 0.8 | 1.5 ± 0.6* | 16.4 ± 6.3** |

Splenocytes and lymph node cells were harvested ex-vivo from G-1 and E2 implanted mice and cultured in a 96-well flat bottom tissue culture plate at 4×10⁵ cells/well in stimulation medium in the presence of APC, irradiated (2500 rad) syngenic thymocytes at a ration of 1:10 (T:APCs) wither with or without mMog 35-55 peptide at varying concentrations. The cells were incubated for 3 days at 37° C. in 7% CO₂ and pulsed with 0.5 μCi of [³H] thymidine (Perkin-Elmer, Boston, Mass.) for the final 18 h of incubation. The cells were harvested onto glass fiber filters, and incorporated radioactivity was measured by a liquid scintillation counter. The cpm values (mean±SD) were calculated from triplicate wells. Stimulation index (SI) was calculated by dividing the experimental cpm by the control cpm.

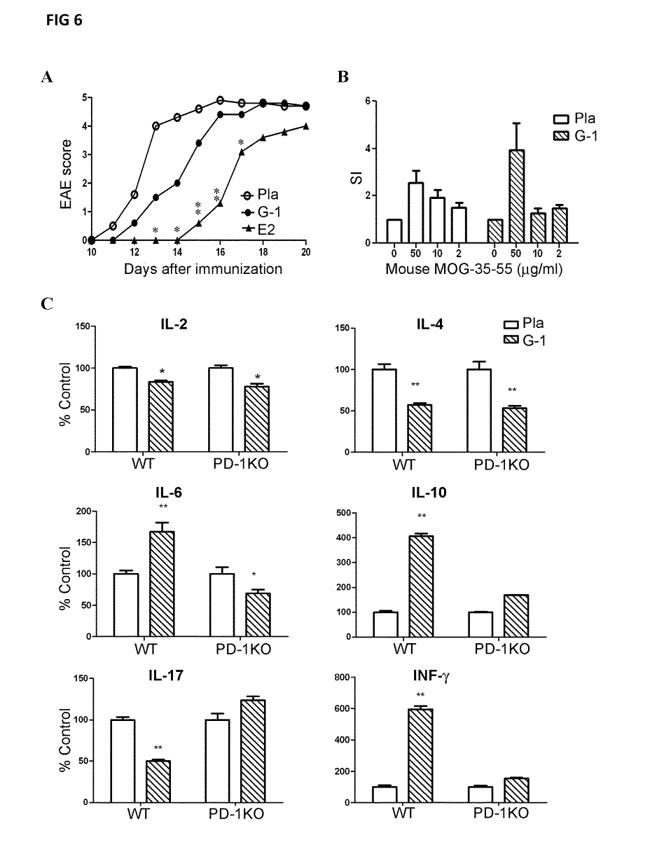

Splenocytes from placebo and G-1 treatment groups were evaluated for T cell proliferation and cytokine secretion in 48 h supernatants. (*P<0.05 or **P<0.01 compared to placebo) As shown in FIG. 6A and Table IV, G-1 failed to protect against clinical EAE in PD-1KO mice, whereas E2 retained partial efficacy.

Table IV. The protective effect of G-1, but not E2, was abolished in PD-1KO mice^{d}.

^{d}PD-1KO mice were immunized one week after implantation with 1.8 mg/40 day release G-1 or 2.5 mg/60 day release E2 pellets for a week. *P<0.05 or **P<0.01 as compared to placebo control.
In addition, G-1 treatment did not significantly change T cell proliferation to mMog-35-55 (FIG. 6B). Thus, upregulation of Pd-1 is of critical importance for G-1 induced EAE protection.

### Example X

Evaluation of the Effects of G-1 Treatment and PD-1 Expression on Cytokine Profiles

PD-1KO (Dr. Tasuku Honjo, Kyoto Univeristy, Kyoto, Japan) mice and WT (National Cancer Institute, Frederick, Md.) mice were implanted with placebo (Innovative Research of America, Sarasota, Fla.), 1.8 mg/40 day release G-1 (Cayman Chemicals, Ann Arbor, Mich., Innovative Research of America, Sarasota, Fla.) or 2.5 mg/60 day release 17β estradiol (E2) pellets (Innovative Research of America, Sarasota, Fla.). One week after implantation experimental autoimmune encephalomyelitis (EAE) was induced.

EAE was induced by inoculating the mice s.c. in the flanks with 0.2 ml of an emulsion containing 200 μg of mMOG-35-55 peptide and an equal volume of complete Freund's adjuvant (CFA) containing 200 μg of heat killed Mycobacterium tuberculosis H37RA (DIFCO, Detroit, Mich.). On the same day and again 2 days after immunization, each mouse was injected i.v. with 75 and 200 ng of pertussis toxin (PTX, List Biological Laboratories, Campbell, Calif.) respectively. The mice were assessed daily for clinical signs of EAE according to the following scale: 0=normal, 1=limp tail or mild hindlimb weakness, 2=moderate hindlimb weakness or mild ataxia, 3=moderately severe hindlimb weakness, 4=severe hindlimb weakness or mild forelimb weakness or moderate ataxia, 5=paraplegia with no more than moderate forelimb weakness, and 6=paraplegia with severe forelimb weakness or severe ataxia, moribund condition or dead. After 20 days, the mice were euthanized. The experiment was repeated 2 times with a total of 7-10 mice in each group.

Splenocytes were harvested from G-1, placebo treated WT and PD-1KO immunized mice and evaluated for T cell proliferation and cytokine secretion in 48 h supernatants. (*P<0.05, or **P<0.01 compared to placebo). Lymph node and spleen cells were cultured at 4×10⁶ cells/well in a 24 well flat-bottom culture plate in stimulation medium (RPMI 1640, 1% sodium pyruvate, 1% _{L}-glutamine, 0.4% 2-β-ME, 10% FBS) with 25 μg/ml mMog-35-55 peptide for 48H. Supernatants were then harvested and stored at −80° C. until tested for cytokines. Culture supernatants were assessed for cytokine levels using a Luminex Bio-Plex mouse cytokine assay kit (BioRad, Hercules, Calif.) following the manufacturer's instructions. The following cytokines were determined in a single assay in three separate experiments: IL-1βIFN-γ, TNF-α, IL-2, IL-4, IL-5. IL-6, IL-12, IL-13, and IL-17. As shown in FIG. 6C, G-1 treatment significantly reduced secretion of IL-17 and IL-2, critical pro-inflammatory cytokines that play a vital role in EAE induction, and increased the secretion of IL-10, a key anti-inflammatory cytokine. G-1 also increased the production of IFN-γ, a hallmark Th1 cytokine, as well as Il-6, and suppressed Il-4, but did not significantly change the production of any other cytokine examined, including IL-1β, IL-5, Il-12 and TNF-α. This cytokine profile was drastically altered in G-1 treated PD-1Ko mice. The decrease of Il-17 and increase of Il-6, Il-10 and IFN-γ production were abolished, whereas the reduction in IL-2 and Il-4 remained. Thus PD-1 was required for DPR30 downregulation of IL-17 but not Il-2 and Il-4, and upregulation of IL-6, Il10 and IFN-γ. The result that G-1 treatment reduced Il-17 production through a PD-1 dependent mechanism is of particular importance because this cytokine has been closely linked to neuroinflammation in EAE.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

1. Whitacre, C. C. 2001. Sex differences in autoimmune disease. Nat. Immunol. 2:777-780.
2. Abramsky, O. 1994. Pregnancy and multiple sclerosis. Ann. Neurol. 36 Suppl:S38-S41.
3. Confavreux, C., Hutchinson, M., Hours, M. M., Cortinovis-Tourniaire, P., and Moreau, T. 1998. Rate of pregnancy-related relapse in multiple sclerosis. Pregnancy in Multiple Sclerosis Group. N. Engl. J. Med. 339:285-291.
4. Sicotte, N. L., Liva, S. M., Klutch, R., Pfeiffer, P., Bouvier, S., Odesa, S., Wu, T. C., and Voskuhl, R. R. 2002. Treatment of multiple sclerosis with the pregnancy hormone estriol. Ann. Neurol. 52:421-428.
5. Soldan, S. S., varez Retuerto, A. I., Sicotte, N. L., and Voskuhl, R. R. 2003. Immune modulation in multiple sclerosis patients treated with the pregnancy hormone estriol. J. Immunol. 171:6267-6274.
6. Bebo, B. F., Jr., Fyfe-Johnson, A., Adlard, K., Beam, A. G., Vandenbark, A. A., and Offner, H. 2001. Low-dose estrogen therapy ameliorates experimental autoimmune encephalomyelitis in two different inbred mouse strains. J. Immunol. 166:2080-2089.
7. Ito, A., Bebo, B. F., Jr., Matejuk, A., Zamora, A., Silverman, M., Fyfe-Johnson, A., and Offner, H. 2001. Estrogen treatment down-regulates TNF-alpha production and reduces the severity of experimental autoimmune encephalomyelitis in cytokine knockout mice. J. Immunol. 167:542-552.
8. Dechering, K., Boersma, C., and Mosselman, S. 2000. Estrogen receptors alpha and beta: two receptors of a kind? Curr. Med. Chem. 7:561-576.
9. Toran-Allerand, C. D., Singh, M., and Setalo, G., Jr. 1999. Novel mechanisms of estrogen action in the brain: new players In an old story. Front Neuroendocrinol. 20:97-121.
10. Revankar, C. M., Cimino, D. F., Sklar, L. A., Arterburn, J. B., and Prossnitz, E. R. 2005. A transmembrane intracellular estrogen receptor mediates rapid cell signaling. Science 307:1625-1630.
11. Prossnitz, E. R., Arterburn, J. B., and Sklar, L. A. 2007. GPR30: A G protein-coupled receptor for estrogen. Mol. Cell Endocrinol. 265-266:138-142.
12. Prossnitz, E. R., Arterburn, J. B., Smith, H. O., Oprea, T. I., Sklar, L. A., and Hathaway, H. J. 2008. Estrogen signaling through the transmembrane G protein-coupled receptor GPR30. Annu. Rev. Physiol 70:165-190.
13. Prossnitz, E. R., Oprea, T. I., Sklar, L. A., and Arterburn, J. B. 2008. The ins and outs of GPR30: A transmembrane estrogen receptor. J. Steroid Biochem. Mol. Biol.
14. Wang, C., Dehghani, B., Magrisso, I. J., Bonhomme, E., Cody, D. B., Elenich, L. A., Subramanian, S., Murphy, S. J., Kelly, M. J. et al. 2008. GPR30 contributes to estrogen-induced thymic atrophy. Mol. Endocrinol. 22:636-648.
15. Polanczyk, M., Zamora, A., Subramanian, S., Matejuk, A., Hess, D. L., Blankenhorn, E. P., Teuscher, C., Vandenbark, A. A., and Offner, H. 2003. The protective effect of 17beta-estradiol on experimental autoimmune encephalomyelitis is mediated through estrogen receptor-alpha. Am J. Pathol. 163:1599-1605.
16. Polanczyk, M. J., Hopke, C., Vandenbark, A. A., and Offner, H. 2007. Treg suppressive activity involves estrogen-dependent expression of programmed death-1 (PD-1). Int. Immunol. 19:337-343.
17. Wang, C., Gold, B. G., Kaler, L. J., Yu, X., Afentoulis, M. E., Burrows, G. G., Vandenbark, A. A., Bourdette, D. N., and Offner,H.2006. Antigen-specific therapy promotes repair of myelin and axonal damage in established EAE. J. Neurochem. 98:1817-1827.
18. Morales, L. B., Loo, K. K., Liu, H. B., Peterson, C., Tiwari-Woodruff, S., and Voskuhl, R. R. 2006. Treatment with an estrogen receptor alpha ligand is neuroprotective in experimental autoimmune encephalomyelitis. J. Neurosci. 26:6823-6833.
19. Bologa, C. G., Revankar, C. M., Edwards, B. S., Arterburn, J. B., Kiselyov, A. S, Parker, M. A. Tkachenko, S. E., Savchuck, N. P., Sklar, L. A. et al. 2006. Virtual and biomolecular screening converge on a selective agonist for GPR30. Nat. Chem. Biol. 2:207-212.
20. Trapp, B. D., Peterson, J., Ransohoff, R. M., Rudiek, R., Mork, S., and Bo, L. 1998. Axonal transection in the lesions of multiple sclerosis. N. Engl. J. Med. 338:278-285.
21. Pitt, D., Werner, P., and Raine, C. S. 2000. Glutamate excitotoxicity in a model of multiple sclerosis. Nat. Med. 6:67-70.
22. Polanczyk, M. J., Carson, B. D., Subramanian, S., Afentoulis, M., Vandenbark, A. A., Ziegler, S. F., and Offner,H.2004. Cutting edge: estrogen drives expansion of the CD4+CD25+ regulatory T cell compartment. J. Immunol. 173:2227-2230.
23. Bettelli, E., Oukka, M., and Kuchroo, V. K. 2007. T(H)-17 cells in the circle of immunity and autoimmunity. Nat. Immunol. 8:345-350.
24. Qiu, J., Bosch, M. A., Tobias, S. C., Krust, A., Graham, S. M., Murphy, S. J., Korach, K. S., Chambon, P., Scanlan, T. S., Ronnekleiv, O. K. et al. 2006. A G-protein-coupled estrogen receptor is involved in hypothalamic control of energy homeostasis. J. Neurosci. 26:5649-5655.
25. Revankar, C. M., Mitchell, H. D., Field, A. S., Burai, R., Corona, C., Ramesh, C., Sklar, L. A., Arterburn, J. B., and Prossnitz, E. R. 2007. Synthetic estrogen derivatives demonstrate the functionality of intracellular GPR30. ACS Chem. Biol. 2:536-544.
26. Keir, M. E., Francisco, L. M., and Sharpe, A. H. 2007. PD-1 and its ligands in T-cell immunity. Curr. Opin. Immunol. 19:309-314.
27. Panitch, H. S., Hirsch, R. L., Schindler, J., and Johnson, K. P. 1987. Treatment of multiple sclerosis with gamma interferon: exacerbations associated with activation of the immune system. Neurology 37:1097-1102.
28. Billiau, A., Heremans, H., Vandekerckhove, F., Dijkmans, R., Sobis, H., Meulepas, E., and Carton, H. 1988. Enhancement of experimental allergic encephalomyelitis in mice by antibodies against IFN-gamma. J. Immunol. 140:1506-1510.
29. Ferber, I. A., Brocke, S., Taylor-Edwards, C., Ridgway, W., Dinisco, C., Steinman, L., Dalton, D., and Fathman, C. G. 1996. Mice with a disrupted IFN-gamma gene are susceptible to the induction of experimental autoimmune encephalomyelitis (EAE). J. Immunol. 156:5-7.
30. Voorthuis, J. A., Uitdehaag, B. M., De Groot, C. J., Goede, P. H., van der Meide, P. H., and Dijkstra, C. D. 1990. Suppression of experimental allergic encephalomyelitis by intraventricular administration of interferon-gamma in Lewis rats. Clin. Exp. Immunol. 81:18:3-188
31. Nalbandian and Kovats, Estrogen, Immunity & Autoimmune Disease Curr. Med. Chem.—Immun., Endoc. & Metab. Agents, 2005, 5, 85-91).
32. Watson and Gametchu, Proteins of Multiple Classes May Participate in Nongenomic Steroid Actions Experimental Biology and Medicine 228:1272-1281 (2003)

We claim:

1. A method for preventing or treating autoimmune disorder comprising activating a membrane estrogen receptor in a mammalian subject.

2. The method of claim 1, wherein the membrane estrogen receptor is GPR30.

3. The method of claim 1, wherein the membrane estrogen receptor is activated by G-1.

4. The method of claim 1, wherein the autoimmune disorder is selected from the group consisting of multiple sclerosis, rheumatoid arthritis, Graves' disease, systemic lupus erythematosus, or Hashimoto's thyroiditis.

5. The method of claim 4, wherein the autoimmune disorder is multiple sclerosis.

6. The method of claim 1, wherein activation of the membrane estrogen receptor is not accompanied by activation of intracellular estrogen receptors.

7. The method of claim 1, wherein activation of the membrane estrogen receptor enhances suppressive activity of CD4⁺Foxp3⁺ Treg cells.

8. The method of claim 1, wherein activation of the membrane estrogen receptor reduces one or more condition(s) associated with the autoimmune disorder selected from immune cell infiltration, demyelination in the central nervous system, and axonal damage.

9. The method of claim 1, wherein activation of the membrane estrogen receptor upregulates programmed death 1 (PD-1) in Treg cells.

10. The method of claim 1, wherein activation of the membrane estrogen receptor decreases Il-17 production.

11. A method for treating or preventing an autoimmune disorder in a mammalian subject comprising administering a membrane estrogen receptor agonist to said subject.

12. The method of claim 11, wherein the membrane estrogen receptor agonist is G-1.

13. The method of claim 11, further comprising administering a secondary autoimmune disorder therapeutic agent that is effective in a combinatorial formulation or coordinate treatment regimen with said membrane estrogen receptor agonist to prevent or treat autoimmune disorders or related symptoms or conditions thereof.

14. The method of claim 11, wherein the autoimmune disorder is selected from the group consisting of multiple sclerosis, rheumatoid arthritis, Graves' disease, systemic lupus erythematosus, or Hashimoto's thyroiditis.

15. The method of claim 11, wherein administration of the membrane estrogen receptor agonist does not result in activation of intracellular estrogen receptors.

16. The method of claim 11, wherein activation of the membrane estrogen receptor agonist enhances suppressive activity of CD4⁺Foxp3⁺ Treg cells.

17. The method of claim 11, wherein administration of the membrane estrogen receptor agonist reduces one or more condition(s) associated with the autoimmune disorder selected from immune cell infiltration, demyelination in the central nervous system, and axonal damage.

18. The method of claim 11, wherein administration of the membrane estrogen receptor agonist upregulates programmed death 1 (PD-1) in Treg cells.

19. The method of claim 11, wherein administration of the membrane estrogen receptor agonist decreases Il-17 production.

20. A composition for preventing or treating an autoimmune disorder in a mammalian subject comprising a membrane estrogen receptor agonist and a secondary autoimmune disorder therapeutic or adjunctive therapeutic agent.
